(19) United States
(12) Patent Application Publication
(10) Pub. No.: US 2013/0210984 A1
(43) Pub. Date: Aug. 15, 2013

(54) Secondary Alcohols and Esters Made Therefrom

(71) Applicants: Karla S. Colle, Magnolia, TX (US); Allen D. Godwin, Seabrook, TX (US)

(72) Inventors: Karla S. Colle, Magnolia, TX (US); Allen D. Godwin, Seabrook, TX (US)

(73) Assignee: ExxonMobil Chemical Patents Inc., Baytown, TX (US)

(21) Appl. No.: 13/704,408

(22) PCT Filed: Jun. 3, 2011
(86) PCT No.: PCT/US2011/039024

§ 371 Date: Feb. 20, 2013

Related U.S. Application Data
(60) Provisional application No. 61/365,210, filed on Jul. 16, 2010.

(51) Int. Cl. C07C 67/00

(52) U.S. Cl. CPC C07C 67/00
USPC 524/315; 560/247

(57) ABSTRACT

The invention relates to secondary alcohols produced by contacting an olefin and a carboxylic acid with a zeolite, esters made therefrom, and to plasticizer compositions comprising the esters.

### CROSS REFERENCE TO RELATED APPLICATIONS

This application claims the benefit of Ser. No. 61/365,210, filed Jul. 16, 2010, and the disclosure of which is incorporated by reference in its entirety.

### FIELD OF THE INVENTION

The invention relates to secondary alcohols produced by contact of an olefin and a carboxylic acid with a zeolite, esters made therefrom, and to plasticizer compositions comprising said esters.

### BACKGROUND OF THE INVENTION

Phthalates and many other esters are well-known as plasticizers for PVC and other plasticizable resins. These esters are commonly produced by esterification of an acid (or anhydride) with a primary alcohol.

Esters may also be derived from secondary alcohols. The effectiveness of secondary alcohol esters as plasticizers; however, is known to be dependent on the position of the hydroxyl group in the alcohol molecule. Secondary alcohol esters with a high degree of hydroxyl content at the second or C₂—OH carbon are considerably more effective than if the hydroxyl group is located more toward the interior of the carbon chain. The use of secondary alcohol esters as plasticizers has been limited by the availability of secondary alcohols with a high C₂—OH content.

The use of esters of secondary alcohols as plasticizers in various polymeric systems has recently been described in WO 2009/070398 and WO 2009/070399.

Most secondary alcohols are commercially produced today by oxidation of paraffins. This process is not suitable for production of secondary alcohols for plasticizer use since it is non-selective and produces a broad mixture of products with the OH group located all along the hydrocarbon backbone. Selective direct hydration of olefins to secondary alcohols is not a viable process for higher olefins.

One route to secondary alcohols with a high degree of C₂—OH is the reaction of an olefin with a carboxylic acid in the presence of a particular type of shape selective zeolite catalysts, as taught in U.S. Pat. Nos. 4,365,084 and 4,461,729. The product or the reaction of olefins with carboxylic acids in the presence of certain zeolite materials produces alpha methyl alkyl carboxylates, which may then be reduced to the corresponding secondary alcohol by known methods. This reaction is conducted at ˜200° C./200 psig (1379 kPa) and gives conversions on the order of 25-30% in 5 hours.

Other background references include Esterification of Alkene with Cerium (IV) Sulfate in Carboxylic Acid, Horiuchi et al., J. Chem. Research (S), 2003, pp. 270-272.

The present inventors have discovered that plasticizing esters can be prepared from secondary alcohols in the plasticizer molecular weight range, i.e., having a carbon content of C₄-C₁₃ with a high degree of C₂—OH, where the secondary alcohols are prepared by the reaction of an olefin with a carboxylic acid in the presence of certain zeolites. The resultant alkyl carboxylate, with a high α-methyl alkyl carboxylate content, can be hydrolyzed to yield the secondary alcohol with a high degree of 2-hydroxy isomer, with very little, if any 3-hydroxy or higher structural isomer by-product. These alcohols can be esterified with dicarboxylic acids or tricarboxylic acids or their anhydrides to produce plasticizing esters for polymeric materials such as poly(vinyl chloride).

### SUMMARY OF THE INVENTION

The present invention is directed to a method for making secondary alcohols comprising reacting an olefin with a carboxylic acid in the presence of a zeolite. The resultant alkyl carboxylate, high in alpha-methylcarboxylate (or α-methyl alkyl carboxylate) content, are hydrolyzed to yield a secondary alcohol, with very little, if any 3-hydroxy or higher structural isomer by-product. Such alcohols are particularly useful in preparing esters useful as plasticizers.

The invention is also directed to secondary alcohols in the plasticizer range (C₄-C₁₃) with a high degree of C₂ attachment of the alcohol group (“high C₂—OH content”).

The invention is also directed to plasticizer compositions containing the aforementioned 2-hydroxy isomer, including compositions further comprising plasticizable resins such as PVC, and to articles containing the thus-plasticized resin.

The invention is also directed to a process comprising reacting an olefin with a carboxylic acid in the presence of a zeolite under predetermined conditions, said zeolite and said conditions suitable to produce a product comprising a mixture of alkyl carboxylate esters, including an α-methylalkyl carboxylate ester and other isomers of said alkyl carboxylate esters, wherein the ratio of said α-methylalkyl carboxylate ester to said other isomers is greater than 1, followed by converting at least a portion of the mixture of the alkyl carboxylate esters to a high C₂—OH content secondary alcohol mixture by a method selected from hydrolysis, hydrogenation, and chemical reduction, with subsequent reaction of the secondary alcohol mixture with dicarboxylic acids or tricarboxylic acids or their anhydrides to produce plasticizing esters.

The invention is still further directed to the use of the plasticizing ester made using the processes above in products and the products thereby.

It is an object of the invention to provide a method of preparing plasticizer-range alcohols having a high C₂—OH content that can readily be converted to plasticizer, particularly useful to plasticize PVC resins.

It is another object of the invention to prepare esters more effective as PVC plasticizers than esters derived from other secondary alcohols due to their high C₂ point of attachment.

These and other objects, features, and advantages will become apparent as reference is made to the following detailed description, preferred embodiments, examples, and appended claims.

### DETAILED DESCRIPTION

According to the invention, secondary alcohols in the plasticizer range (C₄-C₁₃) with a high degree of C₂—OH can be prepared by the reaction of an olefin with a carboxylic acid in the presence of certain zeolites. The resultant alkyl carboxylate, with a high α-methyl alkyl carboxylate content, can be hydrolyzed to yield the secondary alcohol with a high degree of 2-hydroxy isomer, with very little, if any 3-hydroxy or higher structural isomer by-product.

The first step in the process is the reaction of an olefin with a carboxylic acid to selectively produce an alkyl carboxylate ester product enriched in the α-methylalkyl carboxylate ester. The reaction is carried out at moderate temperatures and pressures (˜200° C. and 250 psig (1724 kPa)). By utilization of particular zeolite catalysts (especially ZSM-12), it is possible to react carboxylic acids with olefins having the carbon-carbon double bond in substantially any position in the molecule and to selectively produce an adduct where the carboxylate has attached principally at the C₂ carbon of the molecule. Other non-shape selective catalysts can produce high yields of alkyl carboxylate esters but are not selective for the 2-isomer.

Suitable α-olefins for this process include but not limited to are hexene-1, heptene-1, octene-1, nonene-1, decene-1, undecene-1, dodecene-1, tridecene-1, and tetradecene-1, substituted α-olefins such as 3-methyl heptene-1,4-methyl heptene-1,5-methyl heptene-1,6-methyl heptene-1,3-methyl octene-1,4-methyl octene-1,5-methyl ocetene-1, 6 methyl octene-1,7-methyl octene-1, 3 methyl nonene-1, 4 methyl nonene-1, 5 methyl nonene-1, 6 methyl nonene-1, 7 methyl nonene-1, 8 methyl nonene-1, similar methyl substituted olefins ranging in carbon number from C₅ to C₁₃ or olefin mixtures containing greater than 50% of the C₅-C₁₃ linear α-olefins.

Carboxylic acids used to make the carboxylate esters include formic acid, acetic acid or acetic anhydride, propanoic acid, butyric acid, isobutyric acid, pentanoic acid, isopentanoic acid, neopentanoic acid, hexanoic acid, isohexanoic acid, heptanoic acid, isoheptanoic acid, and 2-ethyl hexanoic acid.

As shown in Table 1 below, alkyl carboxylates are produced with a high α-methyl alkyl carboxylate content, and very little, if any 3-hydroxy or higher structural isomer by-product when an alpha olefin such as octene-1 is used.

TABLE 1

| | | Yield of | | | | |
|---|---|---|---|---|---|---|
| | | C8-0Ac at | | | | Ratio of |
| | | ~2 hrs | 2-C80Ac | 3-C80Ac | 4-C80Ac | Isomers |
| Olefins | Catalyst | (wt % ) | % | % | % | 2/(3 + 4) |
| Octene-1 | ZSM-12 | 23.9 | 94.1 | 5.5 | 0.4 | 15.9 |
| Octene-2 | ZSM-5 | 3.6 | 80 | 17 | 3 | 4.0 |
| | ZSM-12 | 12.9 | 79 | 20 | 0.6 | 3.4 |
| | BF3•Et2O | 57.0 | 52 | 44 | 4 | 1.1 |
| | Amorph | 11.1 | 59 | 36 | 5 | 1.4 |
| | SiO2/A12O3 | | | | | |
| | REY | 1.9 | 59 | 37 | 4 | 1.4 |
| Octene-4 | ZSM-12 | 10.5 | 60 | 30 | 10 | 1.5 |
| | BF3•Et2O | 65.7 | 8 | 18 | 75 | 0.09 |
| | Amorph | 3.5 | 5 | 10 | 85 | 0.05 |
| | SiO2/A12O3 | | | | | |
| | REY | 4.1 | 4 | 7 | 89 | 0.04 |
| Mixed Linear olefins | ZSM-12 | 17.0 | 86 | 13 | 1 | 6.1 |

A range of carboxylic acids may be used, but acetic acid is particularly desirable. One of the advantages of the present invention is that virtually any olefin may be used without regard to the position of the double bond. Mixed isomers of an olefin are particularly desirable given their ready availability and relatively lower cost. Linear olefins may be preferred, but slightly branched olefins are suitable. The catalysts are per se well-known zeolite materials with high Si/Al ratio. Especially good yields with high selectivity to the desired isomer have been obtained using ZSM-12.

The α-methyl alkyl enriched carboxylate ester product of the first step may be hydrolyzed to convert the ester functionality to the corresponding secondary alcohol and allow recovery of the carboxylic acid. The hydrolysis of esters is well-known per se and may be carried out in a number of ways. One method involves ester cleavage under base conditions such as refluxing the carboxylate ester with alcoholic potassium hydroxide or aqueous sodium hydroxide. Other applicable methods to obtain the secondary alcohol would include chemical reduction with reagents such as lithium aluminum hydride or sodium borohydride or hydrogenation over one of a variety of nickel or palladium or platinum hydrogenation catalysts. These methods would not allow recovery of the carboxylic acid however. One of skill in the art in possession of the present disclosure can perform the conversion to the desired product without undue experimentation.

The secondary alcohol may be used to prepare esters, especially phthalate esters or adipate esters or trimellitate esters or citrate esters or terephthalate esters or benzoate esters or cyclohexanoate diesters, and mixtures thereof, which are useful as plasticizers. Without wishing to be bound by theory, these esters are more effective as PVC plasticizers than esters derived from other secondary alcohols due to their high C₂ point of attachment.

Work by J. W. Hayden in Society of Plastics Engineering, Annual Technical Conference, 28th (1970) pp. 46-47, SPE Publisher, Greenwich, Conn., reported that the performance of phthalate esters prepared with secondary alcohols, diminishes significantly as the C₂—OH substitution is changed from the C₂ position to C₃, C₄, and C₅. Secondary alcohol phthalate esters made with C₂—OH rich blends will have very good performance in PVC plasticizer efficiency, low temperature flexibility, lower volatility and lower plastisol viscosity, while other secondary alcohol phthalate esters prepared with reduced on no C₂—OH substituted alcohols, show decreased to unacceptable performance in these areas.

The secondary alcohol from above may be reacted with phthalic anhydride or other acids to produce esters which are especially useful as PVC plasticizers. By way of example and without intending to be limiting, the phthalate ester of 2-octanol can be prepared by reacting 2.2 moles of 2-octanol with 1 mole of phthalic anhydride, using well known esterification conditions, under either a nitrogen atmosphere or an oxygen-free atmosphere obtained through vacuum or through a combination of nitrogen purging and applying a vacuum, using one of a variety of tin or titanium organometallic catalysts.

In a preferred embodiment, the secondary alcohol phthalate may be hydrogenated over ruthenium or nickel catalysts to yield the non-phthalate secondary alcohol cyclohexanoate diester. These esters will be more effective as PVC plasticizers than esters derived from other secondary alcohols due to their high C₂—OH content. Other plasticizing esters can be prepared by reacting these high C₂—OH content alcohols with adipic acid, terephthalic acid, dimethyl terephthalate, trimellitic anydride, citric acid, hexahydrophthalic anhydride, dimethyl cyclohexanedicarboxylic acid ester, or benzoic acid. The secondary alcohols of the C₁₀-C₁₃ carbon number range may also be alkoxylated to produce highly desirable secondary alcohol surfactants.

The plasticizing esters can be used to form flexible PVC compositions. For example, these plasticizers can be mixed with suspension grade PVC resin, in a composition that can range from 20 to 100 parts of plasticizer per hundred parts of PVC resin (parts as used herein are parts by weight). The material is further heated under mixing, using techniques such as Banbury mixers, roll mills, calandars, extruders, or injection molding to produce flexible PVC compositions. The same plasticizers can be used to prepare plastisol compositions by mixing 40 to 100 parts of these plasticizers with 100 parts of a paste or emulsion grade PVC resin. Plastisols can be converted to flexible PVC articles upon heating from 160-200° C. Other additives can be added to these formulations included PVC stabilizers, fillers, secondary plasticizers, lubricants, colorants, pigments, and foaming agents. Applications for such flexible PVC compositions include wire and cable insulation or jacketing, vinyl flooring, PVC backed carpet, automotive underbody sealants, PVC adhesives, wall paper, synthetic leather, toys, shoes, traffic cones, coated fabrics, awnings, tarpaulins, films, tubing, and medical devices. In use with acrylic polymers, these plasticizers can be used to prepare acrylic caulks, adhesives, coatings, and sealants.

Trade names used herein are indicated by a ™ symbol or ® symbol, indicating that the names may be protected by certain trademark rights, e.g., they may be registered trademarks in various jurisdictions. All patents and patent applications, test procedures (such as ASTM methods, UL methods, and the like), and other documents cited herein are fully incorporated by reference to the extent such disclosure is not inconsistent with this invention and for all jurisdictions in which such incorporation is permitted. When numerical lower limits and numerical upper limits are listed herein, ranges from any lower limit to any upper limit are contemplated. While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth herein but rather that the claims be construed as encompassing all the features of patentable novelty which reside in the present invention, including all features which would be treated as equivalents thereof by those skilled in the art to which the invention pertains.

The invention has been described above with reference to numerous embodiments and specific examples. Many variations will suggest themselves to those skilled in this art in light of the above detailed description. All such obvious variations are within the full intended scope of the appended claims.

1. A process comprising reacting an olefin with a carboxylic acid in the presence of a zeolite under predetermined conditions, said zeolite and said conditions suitable to produce a product comprising a mixture of alkyl carboxylate esters, including an α-methylalkyl carboxylate ester and other isomers of said alkyl carboxylate esters, wherein the ratio of said α-methylalkyl carboxylate ester to said other isomers is greater than 1, followed by converting at least a portion of the mixture of the alkyl carboxylate esters to a high C₂—OH content secondary alcohol mixture by a method selected from hydrolysis, hydrogenation, and chemical reduction, with subsequent reaction of the secondary alcohol mixture with dicarboxylic acids or tricarboxylic acids or their anhydrides to produce plasticizing esters.

2. The process of claim 1, wherein said carboxylic acid is selected from C₁-C₅ carboxylic acids.

3. The process of claim 1, wherein said carboxylic acid includes acetic acid.

4. The process of claim 1, wherein said olefin is selected from C₅-C₁₃ alpha olefins or C₅-C₁₃ olefin mixtures containing greater than 50% linear alpha olefins.

5. The process of claim 1, wherein said zeolite is selected from at least one of ZSM-12 and ZSM-5.

6. The process of claim 1, wherein said zeolite is ZSM-12.

7. The process of claim 1, wherein said ratio is greater than 3.

8. The process of claim 1, wherein said ratio is greater than 15.

9. The process of claim 1, wherein said dicarboxylic acid or tricarboxylic acid is selected from adipic acid, phthalic acid, phthalic anhydride, terephthalic acid, dimethyl terephthalic acid, trimellitic acid, trimellitic anhydride, citric acid, hexahydrophthalic acid, hexahydrophthalic anhydride, dimethyl cyclohexanedicarboxylic acid esters, benzoic acid, and mixtures thereof.

10. The process of claim 1, wherein said dicarboxylic acid or tricarboxylic acid is a carboxylic acid with at least one aromatic moiety, said process further characterized by an hydrogenation step wherein said aromatic moiety is converted to a saturated ring compound, either before or after conversion to the plasticizing esters.

11. The process of claim 1, further comprising mixing the resulting plasticizing ester with a polymer in an amount sufficient to plasticize said polymer.

12. The process of claim 11, wherein the polymer is poly(vinyl chloride).

13.-14. (canceled)
